(19) United States
(12) Patent Application Publication
(10) Pub. No.: US 2017/0150894 A1
(43) Pub. Date: Jun. 1, 2017

(54) DEVICE AND SYSTEM FOR PHYSIOLOGICAL MEASUREMENT

(71) Applicant: INDUSTRIAL TECHNOLOGY RESEARCH INSTITUTE, Hsinchu (TW)

(72) Inventors: Hong-Dun Lin, Hsinchu City (TW); Yen-Hsien Lee, Taipei City (TW)

(21) Appl. No.: 15/358,747

(22) Filed: Nov. 22, 2016

(30) Foreign Application Priority Data
Nov. 26, 2015 (TW) 104139473

(51) Int. Cl. A61B 5/021; A61B 5/00; A61B 5/024

(52) U.S. Cl. CPC A61B 5/02108; A61B 5/02433; A61B 5/7275; A61B 5/7225; A61B 5/6826

(57) ABSTRACT

A device and a system for physiological measurements are disclosed. The device includes two light-emitting units and a detection unit. Each one of the two light-emitting units provides a light beam projecting to respective one of two positions of a limb. The detection unit receives the reflected lights from the projected positions of the limb and transfers the received lights out as measuring signals.

### CROSS-REFERENCE TO RELATED APPLICATION

This application is based on, and claims priority from, Taiwan Application Serial No. 104139473, filed on Nov. 26, 2015, the disclosure of which is hereby incorporated by reference herein in its entirety.

### TECHNICAL FIELD

The disclosure relates in general to a device and a system for physiological measurement.

### BACKGROUND

Blood pressure measuring device with electronic resonance cuff and stethoscope has been widely used for measuring the systolic blood pressure and diastolic blood pressure of an artery. During the use of the blood pressure measuring device, the cuff needs to be inflated and deflated so that non-continuous blood pressure can be indirectly measured. When measuring continuous blood pressure, the cuff needs to be correctly installed and repeatedly inflated and deflated in collaboration with an inflating/deflating device. The blood pressure measuring device with electronic resonance cuff and stethoscope has a large volume, takes a long duration of time for measurement, and is inconvenient for user to carry with as a portable device. Thus, the practicability of the blood pressure measuring device with cuff would be significantly decreased.

### SUMMARY

The exemplary embodiments of the disclosure may provide a sensing system for physiology measurements and a sensing method thereof.

According to one embodiment of the disclosure, a device for physiology measurement includes at least two light-emitting units and a detection unit is provided. The two light-emitting units respectively provide a first radiation beam and a second radiation beam irradiated to a first detection position and a second detection position of a body part. The first radiation beam and the second radiation beam are reflected as a first reflected beam and a second reflected beam by the first detection position and the second detection position of the body part. Then, the detection unit receives, converts and outputs the first reflected beam and the second reflected beam as a first detect signal and a second detect signal, respectively.

According to another embodiment of the disclosure, a system for physiology measurement includes a physiological measuring device and a signal processing device is provided. The physiological measuring device is coupled to the signal processing device to exchange information. The physiological measuring device is an integrated unit of at least two light-emitting units and a detection unit. The two light-emitting units respectively provide a first radiation beam and a second radiation beam irradiated to a first detection position and a second detection position of a body part. Then, the first radiation beam and the second radiation beam are reflected by the first detection position and the second detection position of the body part as a first reflected beam and a second reflected beam, respectively. Then, the detection unit receives, converts and outputs the first reflected beam and the second reflected beam as a first detect signal and a detect sensing signal, respectively. The signal processing device receives the first detect signal and the second detect signal outputted from the physiological measuring device.

The above and other aspects of the disclosure will become better understood with regard to the following detailed description of the preferred but non-limiting embodiment (s). The following description is made with reference to the accompanying drawings.

### BRIEF DESCRIPTION OF DRAWINGS

FIG. 1 is a schematic diagram of a device for physiological measurements according to an embodiment;

FIG. 2 is a schematic diagram of a system for physiological measurements according to an embodiment;

FIG. 3 is a schematic diagram of a first pulse wave signal and a second pulse wave signal outputted by a device for physiological measurements according to an embodiment;

FIG. 4 is a schematic diagram illustrating a system structure of a system for physiology measurements according to an embodiment; and

FIG. 5 is an exemplary flowchart of performing blood pressure measurement using a system for physiology measurements according to an embodiment.

### DETAILED DESCRIPTION OF DISCLOSED EMBODIMENTS

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

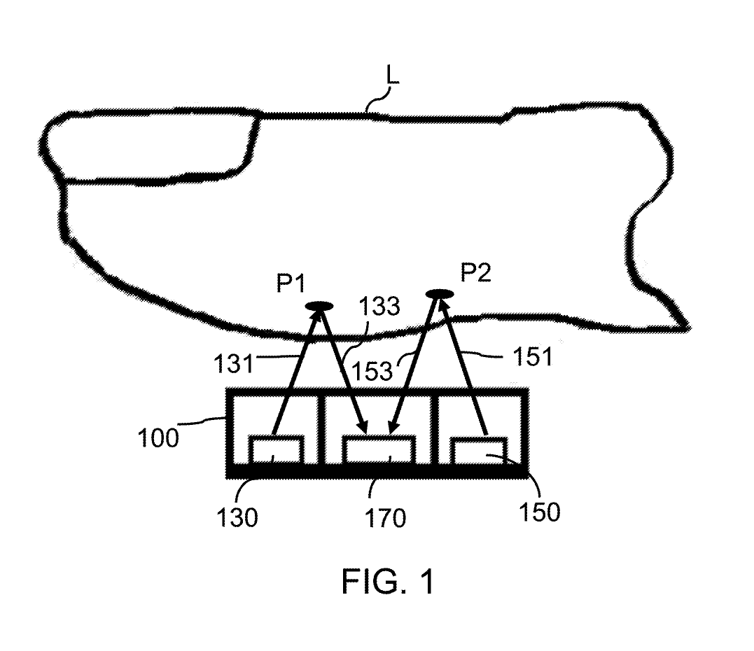

FIG. 1 is a schematic diagram of a device 100 for physiology measurements according to an embodiment. As shown in FIG. 1, the device 100 includes a first light-emitting unit 130, a second light-emitting unit 150 and a detection unit 170. The first light-emitting unit and the second light-emitting unit may be a semiconductor light source, such as a light-emitting diode or a semiconductor laser, but the disclosure is not limited thereto.

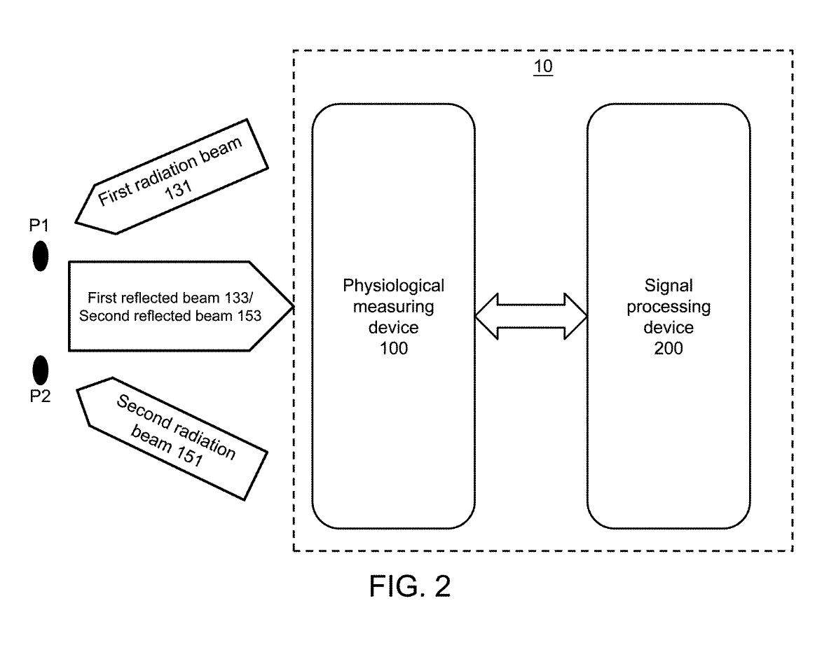

FIG. 2 is a schematic diagram of a system 10 for physiology measurements according to an embodiment. As shown in FIG. 2, the system 10 includes a signal processing device 200 and a physiological measuring device 100. The signal processing device 200 is coupled to the device 100 for physiology measurements by way of wireless communication or signal-line connection to exchange information. Refer to the embodiments shown in FIG. 1 and FIG. 2. The first light-emitting unit 130 and the second light-emitting unit 150 respectively provide a first radiation beam 131 and a second radiation beam 151 to a first detection position P1 and a second detection position P2 of a finger part L of a user. The first radiation beam 131 is reflected to the detection unit 170 as a first reflected beam 133 by the blood vessel 30 at the first detection position P1. Also, the second radiation beam 151 is reflected to the detection unit 170 as a second reflected beam 153 by the blood vessel 30 at the second detection position P2. The first detection position P1 and the second detection position P2 are separated by a distance D. It should be understood that the system for physiology measurements of the present disclosure is not limited to the use of sensing the finger part, and can also be used for sensing other body part of the user, such as the wrist, the upper arm, and the lower arm.

In the present embodiment, the detection unit 170 may be a photodiode, but the disclosure is not limited thereto. Refer to the embodiment shown in FIG. 1. The first light-emitting unit 130, the second light-emitting unit 150 and the detection unit 170 of the device 100 may be an integrated element, such as a package element having light-emitting diode and photodiode therein, but the disclosure is not limited thereto.

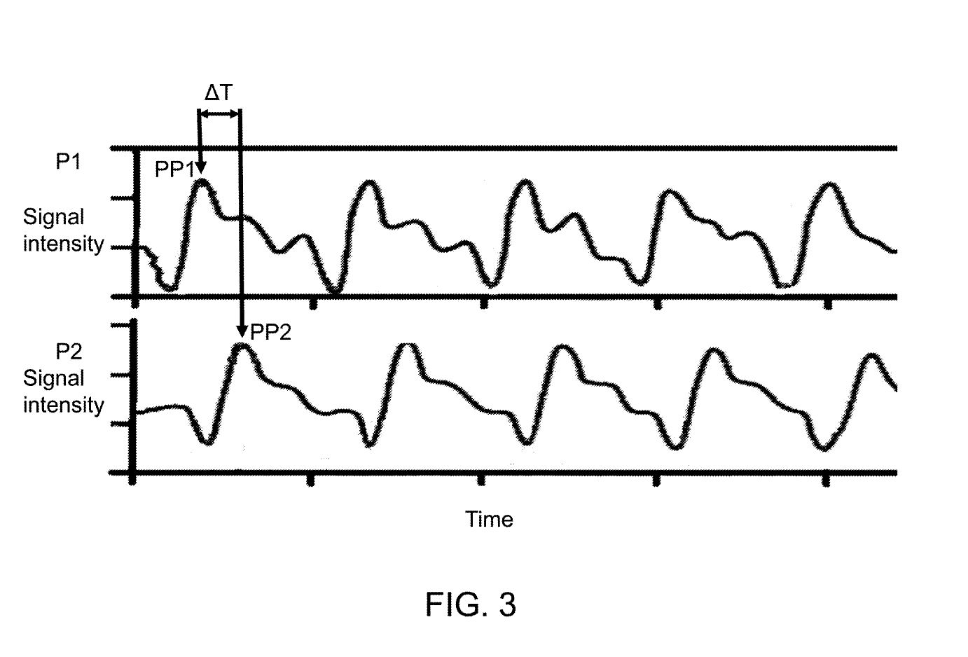

FIG. 3 is a schematic diagram of a first pulse wave signal DS1 and a second pulse wave signal DS2 outputted by a device 100 for physiology measurements according to an embodiment. In the present embodiment, the first pulse wave signal DS1 and the second pulse wave signal DS2 are output signals obtained by performing signal processing on signals, output from the detection unit 170, corresponding to the first reflected beam 133 and the second reflected beam 153 respectively. For example, the first pulse wave signal DS1 and the second pulse wave signal DS2 are signals by filtering or amplifying the signals output from the detection unit 170, but the disclosure is not limited thereto.

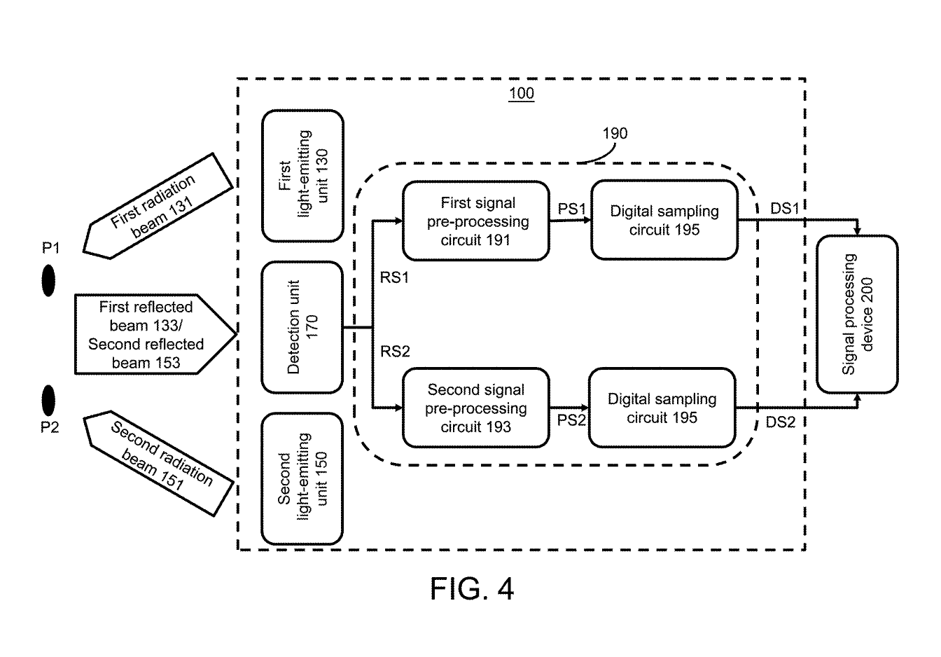

FIG. 4 is a schematic diagram illustrating a system structure of system 10 for physiology measurements according to an embodiment. In the embodiment, a device 100 for physiology measurements of the system 10 further includes a sensing signal processing circuit 190. The detection unit 170 receives and converts a first reflected beam 133 and a second reflected beam 153 into signals, and then outputs the signals as a first sensing signal RS1 and a second sensing signal RS2, respectively. In the present embodiment, the first sensing signal RS1 and the second sensing signal RS2 are processed and outputted as a first pre-processing signal PS1 and a second pre-processing signal PS2 by a first signal pre-processing circuit 191 and a second signal pre-processing circuit 193, respectively. The first signal pre-processing circuit 191 and/or the second signal pre-processing circuit 193 may include a pre-filter circuit, a pre-amplifier circuit, a ban-pass filter (0.8-4 Hz) circuit or a post-amplifier circuit. The digital sampling circuit 195 can process the first pre-processing signal PS1 and the second pre-processing signal PS2 as a first pulse wave signal DS1 and a second pulse wave signal DS2 as shown in FIG. 3 and further output the first pulse wave signal DS1 and the second pulse wave signal DS2 to the signal processing device 200.

According to an embodiment, the sensing system can be used for measuring the pressures of the vessels, such as the systolic blood pressure and diastolic blood pressure of an artery, but the disclosure is not limited thereto. Since the time points at which a blood flow reaches a first detection position P1 and a second detection position P2 of a detection position vessel are different, the generation time of the first pulse wave signal DS1 is different from the generation time of the second pulse wave signal DS2. Refer to FIG. 3, thus a time interval ΔT exists between a first pulse peak PP1 of the first pulse wave signal DS1 and a second pulse peak PP2 of the second pulse wave signal DS2, the pulse wave velocity (PWV) of blood flow can be obtained from the distance D between the two detection positions P1, P2 and the time interval ΔT. The relationship can be expressed as the following formula:

PWV=D/ΔT (1)

Then, the blood pressure (BP) and the pulse wave velocity (PWV) can be obtained from the following formula of the relationship between the blood pressure (BP) and the pulse wave velocity:

BP_{Sys}=a₁×PWV+b₁

BP_{Dia}=a₂×PWV+b₂ (2)

Wherein, BP_{sys} and BP_{Dia} respectively represent a systolic blood pressure and a diastolic blood pressure; parameters a1, a2, b1 and b2 can be obtained by non-linear fitting calculation.

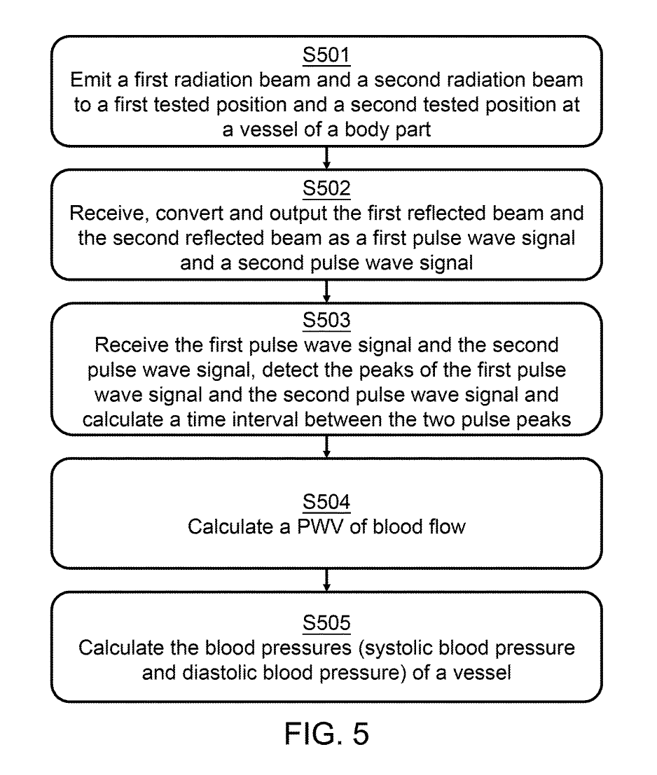

FIG. 5 is an exemplary flowchart of performing blood pressure measurement using a system 10 for physiological measurements according to an embodiment. As shown in FIG. 5, in step S501, the first light-emitting unit 130 and the second light-emitting unit 150 of the device 100 for physiological measurements are controlled by a first controller (not illustrated) to emit a first radiation beam 131 and a second radiation beam 151 at a time interval (an emitting time interval), so that the first radiation beam 131 and the second radiation beam 151 are respectively irradiated to a first detection position P1 and a second detection position P2 of a user's body part. In an embodiment shown in FIG. 2, the first controller may be realized by such as the signal processing device 200, but the disclosure is not limited thereto.

In step S502, the time interval is adjusted by a second controller (not illustrated) such that a detection unit 170 of the physiological measuring device 100 continuously receives a first reflected beam 133 and a second reflected beam 153 reflected from the first detection position P1 and the second detection position P2. After the first reflected beam 133 and the second reflected beam 153 are received by the detection unit 170, the first reflected beam 133 and the second reflected beam 153 are converted and outputted as a first pulse wave signal DS1 and a second pulse wave signal DS2 by the physiological measuring device 100. In an embodiment as shown in FIG. 2, the second controller may be realized by such as the signal processing device 200, but the disclosure is not limited thereto.

In step S503, the first pulse wave signal DS1 and the second pulse wave signal DS2 are received by the signal processing device 200 which further detects the peaks of the first pulse wave signal DS1 and the second pulse wave signal DS2 and obtains a time interval ΔT between the two pulse peaks of the first pulse wave signal DS1 and the second pulse wave signal DS2.

Then, in step S504, a pulse wave velocity of blood flow is obtained with the pulse peak time interval ΔT. In step S505, the blood pressures (systolic blood pressure and/or diastolic blood pressure) at a detected body part are obtained.

In an embodiment, the first light-emitting unit 130 and the second light-emitting unit 150 of the device 100 may respectively emit radiation beams of different wavelengths. For example, wavelengths of the first radiation beam 131 the second radiation beam 151 may respectively be 600 nm and 900 nm, but the disclosure is not limited thereto.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A device for physiology measurement, comprising:
at least two light-emitting units configured to provide a first radiation beam and a second radiation beam to a first detection position and a second detection position of a user's body part respectively; and
a detection unit configured to receive and process a first reflected beam and a second reflected beam to output a first detect signal and a second detect signal respectively, wherein the first radiation beam and the second radiation beam are reflected as the first reflected beam and the second reflected beam by the first detection position and the second detection position of the body part respectively;
wherein the first detect signal and the second detect signal are a first pulse wave signal and a second pulse wave signal respectively.

2. The device as claimed in claim 1, wherein a wavelength of the first radiation beam is different from a wavelength of the second radiation beam.

3. The device as claimed in claim 1, wherein a wavelength of the first radiation beam is 660 nm.

4. The device as claimed in claim 1, wherein a wavelength of the second radiation beam is 900 nm.

5. The device as claimed in claim 1, wherein the device further comprising:
a controller configured to control the two light-emitting units to emit the first radiation beam and the second radiation beam at a time interval.

6. The device as claimed in claim 1, wherein the device further comprises:
a controller configured to adjust a time interval, such that the detection unit continuously receives the first reflected beam and the second reflected beam at the time interval.

7. The device as claimed in claim 1, wherein the device further comprises:
a signal processing circuit configured to receive a first sensing signal and a second sensing signal, process the first sensing signal and the second sensing signal to output as the first detect signal and the second detect signal respectively.

8. The device as claimed in claim 7, wherein the signal processing circuit comprises a filter circuit and an amplifier circuit.

9. The device as claimed in claim 8, wherein the signal processing circuit comprises a ban-pass filter circuit, and a band-pass filtering band of the band-pass filter circuit ranges between 0.8 Hz and 4 Hz.

10. A system for physiology measurement, comprising:
a physiology measurement device, including:
at least two light-emitting units for providing a first radiation beam and a second radiation beam to a first detection position and a second detection position of a user's body part respectively;
a detection unit configured to receive and process a first reflected beam and a second reflected beam to a first detect signal and a second detect signal respectively, wherein the first radiation beam and the second radiation beam are reflected as the first reflected beam and the second reflected beam by the first detection position and the second detection position of the body part respectively; and
a signal processing device configured to receive the first detect signal and the second detect signal out from the physiology measurement device;
wherein the first detect signal and the second detect signal are respectively a first pulse wave signal and a second pulse wave signal.

11. The system as claimed in claim 10, wherein the signal processing device is configured to perform a signal-peak detection to obtain a time-interval information between a signal peak of the first pulse wave signal and a signal peak of the second pulse wave signal.

12. The system as claimed in claim 11, wherein the signal processing device is configured to obtain a systolic blood pressure or a diastolic blood pressure at the user's body part by performing calculations with the time interval information.

13. The system as claimed in claim 10, wherein the signal processing device is configured to receive the first detect signal and the second detect signal from the physiology measurement device by way of wireless communication.
